(19) United States
(12) Patent Application Publication
(10) Pub. No.: US 2002/0098521 A1
(43) Pub. Date: Jul. 25, 2002

(54) Method and marker for the isolation of human multipotent hematopoietic stem cells

(76) Inventors: Dominique Bonnet, Finsbury (GB); Guenahel H. Danet, Philadelphia, PA (US)

Correspondence Address: DANN DORFMAN HERRELL & SKILLMAN, SUITE 720, 1601 MARKET STREET, PHILADELPHIA, PA, 19103-2307, US

(21) Appl. No.: 09/982,473

(22) Filed: Oct. 18, 2001

Related U.S. Application Data
(60) Provisional application No. 60/241,253, filed on Oct. 18, 2000.

(51) Int. Cl. G01N 33/53; G01N 33/567; C12N 5/08

(52) U.S. Cl. 435/7.2; 435/372

(57) ABSTRACT

The present invention provides a human hematopoietic stem cell surface marker, C1qRₚ, which is present on both CD34⁻ and CD34⁺ stem cells. This marker can be used to advantage in methods for the positive selection of both CD34⁻ and CD34⁺ stem cell populations.

This application claims priority under 35 U.S.C. §119(e) to U.S. Provisional Application No. 60/241,253 filed Oct. 18, 2000, the entire disclosure of which is incorporated by reference herein.

### FIELD OF THE INVENTION

This invention relates to the isolation and purification of human stem cells, and more specifically, the invention is directed to a method and marker for the isolation and purification of human multipotent hematopoietic stem cells which may be used to advantage in medical procedures such as bone marrow transplants and liver cell repopulation.

### BACKGROUND OF THE INVENTION

Several publications are referenced by numerals in parentheses in order to more fully describe the state of the art to which this invention pertains. Full citations for these references can be found at the end of the specification. The disclosure of each of these publications is incorporated by reference herein.

Mammalian blood cells may be divided into at least three distinct lineages. These include the lymphoid, myeloid and erythroid cell lineages. The lymphoid lineage comprises B-cells and T-cells which regulate the cellular immune system, provide for the production of antibodies as well as for the detection of foreign agents and/or cells in the blood. The myeloid lineage includes monocytes, granulocytes and megakaryocytes which function to monitor the blood stream for the presence of foreign bodies thereby providing protection against neoplastic cells. The erythroid lineage comprises the red blood cells which act as oxygen carriers. Despite the extraordinarily diverse range in nature, morphology, characteristics and function of blood cells, it is believed that these cells are produced from hematopoietic stem cells that are capable of self regeneration and, following exposure to growth factors, become dedicated to a specific blood cell lineage.

In general, the stem cell population constitutes a very small portion of the total number of leukocytes in bone marrow. Because these cells are less prevalent, the ability to isolate and purify stem cells is more difficult. Unfortunately, the isolation and study of hematopoietic stem cells has been greatly impaired by the lack of known cellular markers expressed on all hematopoietic stem cells.

The use of hematopoietic stem cells and their progeny through bone marrow transplants to reconstitute the hematopoietic system has been employed to treat various blood-related diseases and disorders, such as aplastic anemia, immune deficiencies and several forms of cancer including lymphomas and leukemias (see review in Lu et al. Critical Rev.Oncol/Hematol. 22:61-78 (1996)). Bone marrow transplantation is most commonly used in an attempt to restore hematopoietic function following exposure to myeloablative agents, for example after radiation therapy or chemotherapy in the treatment of a variety of cancers. These therapies, in addition to destroying the cancer, can also result in myelosuppression or myeloablation which, in turn, can lead to infection, bleeding disorders, and other complications. Recent estimate have indicated that the need for transplantation of bone marrow-derived hematopoietic stem cells is growing at a rate of 20% per year and the market for bone marrow derived hematopoietic stem cells is approximately $500 million per year (Strickland, D. Bioworld Today 8(14):1).

Stem cells used for transplantation purposes may be autologous in origin, i.e., they have been isolated from the patient prior to undergoing the transplant. Alternatively, they may be obtained from a donor sharing the appropriate histocompatability antigens with the recipient.

Regardless of the source of stem cells so isolated, a critical need exists for a more efficient and selective isolation procedure to obtain appropriate stem cell populations with suitable regenerative capacity for repopulation of the hematopoietic system in stem cell transplant patients.

### SUMMARY OF THE INVENTION

In accordance with the present invention, a method and cellular marker are provided for the isolation and purification of a stem cell population having superior engraftment capacity. In one embodiment of the invention, the stem cell marker, C1qRₚ, which is present on both CD34⁺ and CD34⁻ stem cells, is used to advantage to isolate both cell populations. Thus, the availability of anti-C1qRₚ antibodies provides a new means to positively select these cells from mixed cell samples.

In a further aspect of the invention, it has been discovered that human Lin⁻CD34⁻CD38⁻C1qRₚ⁺ cells efficiently repopulate the bone marrow of NOD/SCID mice. Accordingly, these cells are suitable for the reconstitution of human bone marrow under appropriate conditions.

The human stem cells so isolated have utility in a variety of protocols. They may be used to advantage to regenerate the hematopoietic system of a host deficient in stem cells. They may also be administered to a host that is diseased following removal of bone marrow. In this aspect, stem cells are removed, the patient is treated with drugs or radiation following which, the regenerating stem cells are re-introduced into the patient. The isolated stem cells of the invention may be used to advantage for research purposes to further detect and evaluate specific growth factors which stimulate the differentiation and/or self-regeneration of stem cells. In addition, these stem cells may be utilized in the treatment of genetic diseases through gene replacement in autologous stem cells.

In yet another aspect of the invention, the present inventors have discovered the that C1qRₚ⁺ stem cells of the invention differentiate into functional hepatocytes. Accordingly, the stem cells of the invention may be used to advantage in methods for the repopulation of the liver due to injury or disease.

### BRIEF DESCRIPTION OF DRAWINGS

FIGS. 1A-1D are scatter plots obtained from FACS analysis of a human umbilical cord blood sample. C1qRₚ⁺ cells are present in the lymphocyte-blast (FIG. 1B), granulocyte (FIG. 1C) and monocyte (FIG. 1D) populations.

FIGS. 2A and 2B show the results of FACS analysis of an enriched C1qRₚ⁺ cell population following removal of cells expressing lineage specific markers (CD2, CD3, CD14, CD16, CD19, CD24, CD56, CD66b, CD41, Glycophorin A) by positive immuno-magnetic selection (FIG. 2A). The side scatter of the sample is shown in FIG. 2B.

FIGS. 3A and 3B are scatter plots of the lineage depleted mononuclear cells of FIG. 2 gated on CD38⁻ cells. The side scatter is shown in FIG. 3B.

FIG. 4 is a graph indicating the levels of human cell engraftment in NOD/SCID mice transplanted with umbilical cord blood Lin⁻CD34⁻CD38⁻C1qRₚ⁺ versus Lin⁻CD34⁻CD38⁻C1qRₚ⁻ cell populations. The level of human engraftment was estimated using both Southern blot analysis and flow cytometry.

FIGS. 5A-5H are scatter plots obtained following four-color FACS analysis of one representative experiment from a pool of four umbilical cord blood samples. To determine whether other marker molecules are associated with C1qRₚ reactivity, cells were stained with fluorescent markers for FLT-3 (FIG. 5A); CD117 (FIG. 5B); KDR (FIG. 5C); AC133 (FIG. 5D); HLA-DR (FIG. 5E); CD90 (FIG. 5F); CXCR4 (FIG. 5G); and CD162 (FIG. 5H).

FIGS. 6A-6H are histograms showing the results of four-color FACS analysis on the cells depicted in FIG. 5 gated on Lin⁻CD34⁻CD38⁻C1qRₚ⁺ cells.

FIGS. 7A-7H are histograms showing the results of four-color FACS analysis on the cells depicted in FIG. 5 gated on Lin⁻CD34⁺Cd38⁻C1qRₚ⁺ cells.

FIGS. 8A-8J are micrographs of liver sections from non-injected NOD/SCID mice immuno-stained for human hepatocyte-specific antigen (FIGS. 8c-8f) or the human hepatocyte growth factor receptor, c-met (FIGS. 8i and 8j).

FIG. 9 shows the resolution of human hepatocyte-specific gene products from human albumin-specific RT-PCR reactions on an SDS polyacrylamide gel.

FIG. 10 is a scatter plot showing a rare population (≦0.1%) of human cells that express MHC class I molecules that lack CD45.

### DETAILED DESCRIPTION OF THE INVENTION

A novel population of primitive human cells lacking both lineage markers and CD34 expression (Lin⁻CD34⁻CD38⁻) has been identified which contains stem cells capable of repopulating NOD/SCID mice or fetal sheep (9-14). The identification and isolation of these cells has been impaired in part because these cells lack widely identified cellular markers, such as Thy-1, HLA-DR and CD38. To date, only negative selection procedures have been employed to purify these cells.

In accordance with the present invention, a stem cell marker, C1qRₚ, has been identified which is expressed on both CD34⁺ and CD34⁻ cells. Accordingly, agents which recognize C1qRₚ may be used to advantage for the positive selection of primitive hematopoietic stem cells.

The ability to isolate and purify stem cells using anti-C1qRₚ will be extremely useful in bone marrow transplantation, as well as transplantation of other organs in association with the transplantation of bone marrow. Stem cells are also important targets for gene therapy, where the inserted genes promote the health of the individual into whom the stem cells are transplanted. Moreover, the ability to isolate stem cells may serve in the treatment of lymphomas and leukemias, as well as other neoplastic conditions such as breast cancer.

In mice, the AA4.1 monoclonal antibody has been used to identify early multipotent hematopoietic stem cells. The cell surface marker recognized by AA4.1 has been cloned and is identical to the murine C1qRₚ, a receptor for the complement C1q molecule (15). Human C1qRₚ is a highly glycosylated transmembrane protein that is the receptor of the complement C1q molecule and in vitro mediates enhancement of Fc and C3b-mediated phagocytosis (16). This molecule has been identified in differentiated myeloid and endothelial cells, however, C1qRₚ expression on human hematopoietic stem cells has not yet been described.

As described in detail below, it has now been discovered that C1qRₚ is expressed on both CD34⁻ and CD34⁺ human hematopoietic stem cells which have differing capacities for repopulating the hematopoietic system. Thus, in accordance with the present invention, a method is provided for the isolation of human Lin⁻CD34⁻CD38⁻C1qRₚ⁺ cells which are highly enriched in CD34^{neg}-NOD/SCID repopulating cells (CD34^{neg}SRC).

The presence of C1qRₚ on the surface of both CD34⁺ and CD34⁻ hematopoietic stem cells indicates that this marker may be used to advantage to isolate both stem cell populations. Conventionally, human hematopoietic stem cells are isolated by either positive selection of cells expressing CD34 (for CD34^{pos}SRC) or by negative depletion of all cells expressing lineage markers or CD34 for isolation of CD34^{neg}SRC. The use of anti-C1qRₚ for the positive selection of essentially all repopulating cells provides significant improvement over current methods and facilitates subsequent further sub-fractionation of the C1qRₚ-expressing cells (based on CD34 or other markers) to isolate desired cell types.

The precise relationship of CD34⁻ and CD34⁺ stem cells to each other is not well understood. In the mouse, there have been conflicting reports suggesting that long-term repopulating cells were only present in the CD34⁻ cell fraction, while others found long-term repopulating cells in both the CD34⁺ and CD34⁻ fraction (12, 17-20). Additionally, cell surface expression of CD34 on stem cells can be modulated by in vivo exposure to 5-Fluorouracil treatment or by in vitro exposure to cytokines. Whether this modulation corresponds to cell activation or induction of cell cycling is unclear. It has been suggested that CD34 protein cycles between the cytoplasm and the cell surface under these stimuli (19). However, this has yet to be conclusively proven.

Using the NOD/SCID mouse model, it was reported that Lin⁻CD34⁻CD38⁻ cells did not possess any activity in standard CFC and LTC-IC assays but did contain repopulating cells, termed CD34^{neg}-SRC (14). The hallmark of this new class of hematopoietic repopulating cells is the absence of classical stem cell-associated cell surface markers such as CD38, HLA-DR, and Thy-1, as well as distinct survival and proliferation responses following in vitro stimulation with a cocktail of cytokines. These differences in phenotype and cytokine responsiveness eliminate any possibility that the human engraftment resulting from Lin⁻CD34⁻CD38⁻ cell transplants was initiated by CD34^{pos}SRC (Lin⁻CD34⁺CD38₋) cells that may have been inadvertently co-purified within the Lin⁻CD34⁻CD38⁻ subfraction.

Monoclonal antibodies directed against C1qRₚ are available and may be used to advantage in methods of the invention for the isolation and characterization of C1qRₚ⁺ cells which may be either CD34⁻ or CD34⁺. Kits incorporating fluorochrome-conjugated antibodies and kits for magnetic cell sorting of C1qRₚ⁺ cells are also within the scope of the present invention.

I. Definitions:

The following definitions are provided to facilitate an understanding of the present invention:

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2 and F(v).

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples and they do not need to be listed here. Further, the term “specific binding pair” is also applicable where either or both of the specific binding member and the binding partner comprise a part of a large molecule.

A “stem cell marker” is a molecule present on the surface of stem cells which can be used to identify and isolate stem cells. This marker can be a protein, glycoprotein, or transmembrane protein. Representative stem cell markers include, but are not limited to, FLT-3, CD117, KDR, AC133, HLA-DR, CD38, CD90, CXCR4, CD162 and Thy-1.

II. Materials and Methods:

The following protocols are provided to facilitate the practice of Examples 1 through 4.

Phenotypic Analysis of Bone Marrow and Umbilical Cord:

Light density mononuclear cells were isolated from human bone marrow and umbilical cord blood and stained with an anti-C1qRₚ biotinylated monoclonal antibody (clone R3, gift from Dr. Andrea Tenner) for 20 minutes, washed twice in PBS+5% FCS and then incubated with anti-CD34 conjugated to fluorescein isothiocyanate (FITC) and anti-CD38 conjugated to phycoerythrin (PE) (Becton Dickinson, San Jose, Calif.); and Streptavidin-PE (Immunotech) for another 20 minutes. An aliquot of cells was also stained with mouse IgG conjugated to FITC and Streptavidin-PE, as an isotype control. Cells were then washed twice in PBS+5% FCS, analyzed on an Epics Elite Cell Sorter (Coulter) and histograms were prepared using FlowJo software.

Lineage Depletion for Isolation of Lin⁻ Cells:

Mononuclear cells were stained with a mixture of lineage-specific antibodies (CD2, CD3, CD14, CD16, CD19, CD24, CD56, CD66b, CD41, Glycophorin A; Stem Cell Technology, Vancouver, Canada), followed by addition of secondary antibody conjugated to metal colloid. Cells were then eluted through a magnetized column to enrich for cells not expressing lineage markers (Lin⁻ ); The enriched Lin⁻ cells were then stained with anti-C1qRₚ biotinylated antibody, followed by anti-human CD34-FITC, Streptavidin-PE and anti-CD38-Allophycocyanin (APC).

Clonogenic Assays:

Purified cells were plated in methylcellulose assays under standard conditions. Briefly, 800 to 2,000 purified cells were plated in methylcellulose cultures (HP4331, Stem Cell Technology) aliquoted in 1 ml volumes in 35 mm suspension culture dishes and incubated at 37° C. After 10 to 14 days, clonogenic progenitors were scored according to standard criteria.

Long-term culture-initiating cells (LTC-IC) were then established according to previously described methods. Sorted cells were incubated on murine stromal cells (M210B4, Stem Cell Technology) and cultured in Human Long-Term Bone Marrow Culture Media (Stem Cell Technology) with hydrocortisone. Cultures were initiated in limiting dilution (3 to 6 replicate wells per dilution) in 96-well plates, where 500-10,000 Lin⁻CD34⁻CD38⁻ +/− C1qRₚ cells/well, were plated. After 5 weeks of incubation, the ability of cultured cells to produce clonogenic progenitors was assessed by plating the entire contents of the individual wells in methylcellulose assays. Wells that contained at least one clonogenic progenitor were scored as positive.

Repopulating Assay:

Purified cell populations at the indicated dose were transplanted by tail vein injection into sublethally irradiated mice (375 cGy using a ¹³⁷Cs-irradiator) according to standard protocol (14). Mice received alternate-day intraperitoneal injections of human cytokines (10 μg of human SCF, 10 μg of human interleukin-3 and 10 μg of human granulocyte-macrophage colony-stimulating factor (PeproTech, Rocky Hill, N.J.) for the first two weeks. Mice were sacrificed 8 to 12 weeks post-transplant and the bone marrow from the femurs, tibiae and iliac crests of each mouse were flushed into IMDM containing 10% FCS.

Analysis of Human Cell Engraftment in Transplanted Mice:

Genomic DNA was isolated from the bone marrow of transplanted mice by standard extraction protocols. EcoRI-digested DNA was separated by agarose gel electrophoresis, transferred onto a positively charged nylon membrane, and probed with a labeled human chromosome 17-specific-satellite probe (p17H8). The level of human cell engraftment was determined by comparing the characteristic 2.7 kb band with those of human/mouse DNA mixtures as controls (limit of detection 0.05% human DNA).

Multiparameter FACS Analysis:

In order to determine the presence of other stem cell markers and/or homing molecules on Lin⁻CD34⁻CD38⁻C1qRₚ⁺ and Lin⁻CD34⁺CD38⁻C1qRₚ⁺ cell populations, a four-color FACS analysis was performed. Cells were stained with KDR, CD162, CD117-PE, CD90-PE, CXC-R4-PE, Flt-3-PE, AC133-PE, HLA-DR-PE, CD34-APC, CD38-PECy5 and biotinylated C1qRₚ. KDR and CD162, two unconjugated antibodies, were identified using a goat anti-mouse-PE. An isotype containing IG-G1-FITC, PE, APC and PECy5 was used as a control.

The following examples provide illustrative methods of practicing the instant invention, and are not intended to limit the scope of the invention in any way.

### EXAMPLE 1

### Presence of C1qRₚ Expressing Cells in Human Bone Marrow and Umbilical Cord Blood

The identification of C1qRₚ as a new marker for the most primitive human hematopoietic stem cells provides a new means by which to further isolate and characterize this cell population. With such a marker, enrichment of pluripotent CD34⁻ stem cells, which is desirable for their use in the clinic, is now possible. Furthermore, the fact that more than 99.5% of Lin⁻CD34⁺CD38⁻ cells co-express C1qRₚ; indicates that a positive selection procedure based on C1qRₚ expression will allow the isolation of all hematopoietic stem cells (both CD34⁻ and CD34⁺).

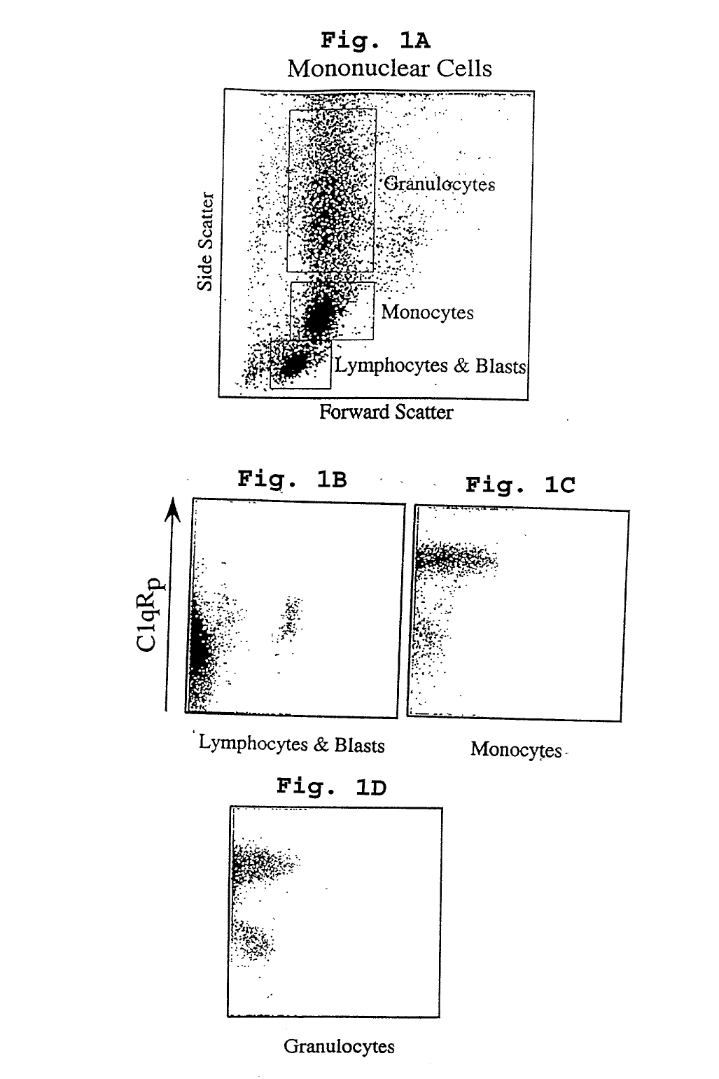

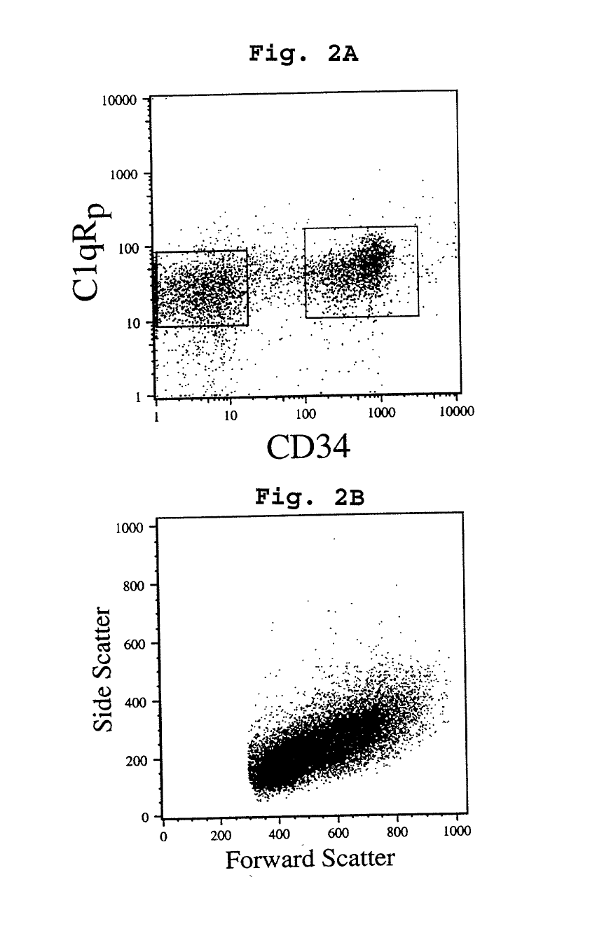

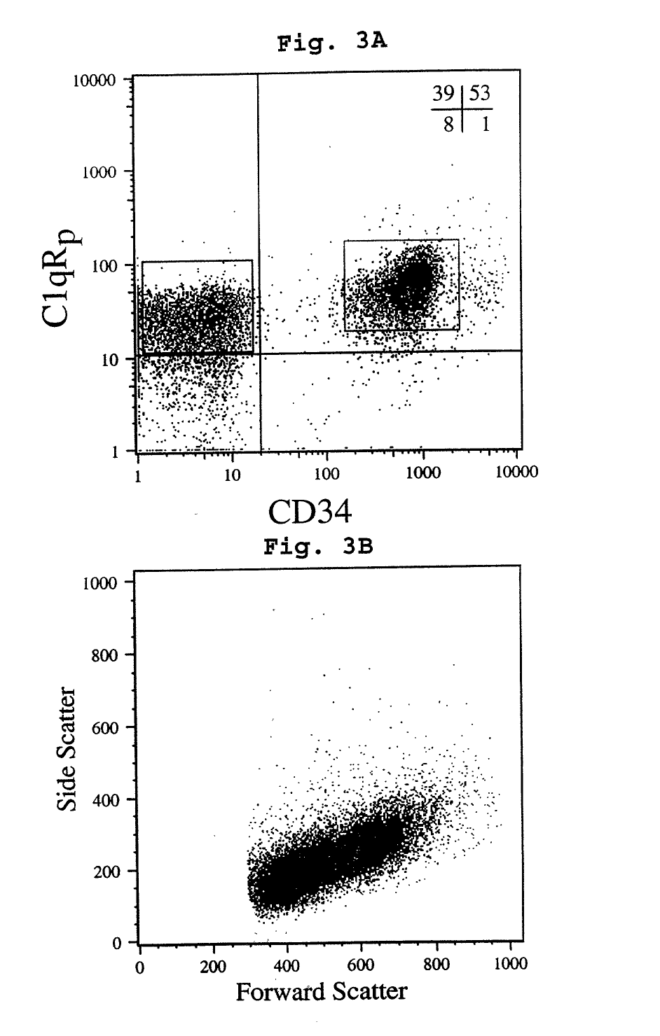

FIGS. 1A-1D show the results of FACS analysis of a human umbilical cord blood sample. C1qRₚ⁺ cells were present in the lymphocyte-blast (FIG. 1B), granulocyte (FIG. 1C) and monocyte (FIG. 1D) populations. The same results were obtained from human bone marrow samples (data not shown). In order to enrich for more primitive cells (e.g., cells not expressing lineage markers; designated Lin⁻), a depletion of committed cells was performed. The enriched Lin⁻ cell fraction included a large proportion of cells that expressed both CD34 antigen and C1qRₚ (FIG. 2A). Interestingly, further examination of the forward and side scatter revealed that the Lin⁻ CD34⁻ C1qRₚ⁺ cell population was smaller than the Lin⁻CD34⁺C1qRₚ⁺ cell population (FIG. 2B). These cell populations were then gated on CD38⁻ cells. Approximately 25 to 40% of the gated Lin⁻CD34⁻CD38⁻ cells expressed C1qRₚ⁺, while 99% of the Lin⁻CD34⁺CD38⁻ cells expressed C1qRₚ⁺ (FIGS. 3A and 3B).

### EXAMPLE 2

### Functional Activity of C1qRₚ⁺ Cells

I. Clonogenic Assays:

The clonogenic capacity of purified Lin⁻CD34⁻CD38⁻C1qRₚ⁺, Lin⁻CD34⁻CD38⁻C1qRₚ⁻, Lin⁻CD34⁺CD38⁻C1qRₚ⁺ and Lin⁻CD34⁺CD38⁻C1qRₚ⁻ cell populations were determined by standard CFC assays. Both Lin⁻CD34⁻CD38⁻C1qRₚ⁺ and Lin⁻CD34⁻CD38⁻C1qRₚ⁻ fractions had a low plating efficiency (PE) with only 1 CFC in 2,000 plated cells and 1 CFC in 3,125 cells, respectively (Table 1A). Interestingly, myeloid (CFU-G, M and GM) and erythroid (BFU-E) colonies were present in both fractions. The presence of these cells indicated that the purification of stem cells based on C1qRₚ expression did not skew the differentiation pattern of the Lin⁻CD34⁻CD38⁻ cell population. In addition, both Lin⁻CD34⁻CD38⁻C1qRₚ⁺ and Lin⁻CD34⁻CD38⁻C1qRₚ⁻ cell populations had a very low frequency in long-term culture-initiating cells (LTC-IC) (Table 1B).

TABLE 1A
Number of CFC progenitors

| CB (4 donors) | CFC/10,000 cells | Plating Efficiency |
|---|---|---|
| Lin⁻CD34⁻CD38⁻ C1qRp⁻ | 3.2 ± 0.2 (n = 7) | 1/3,125 |
| Lin⁻CD34⁻CD38⁻ C1qRp⁺ | 5 ± 3.6 (n = 4) | 1/2,000 |
| Lin⁻CD34⁺CD38⁻ C1qRp⁺ | 3,300 ± 17 (n = 2) | 1/3 |
| Lin⁻CD34⁺CD38⁻ C1qRp⁻ | 2,500 ± 12 (n = 2) | 1/4 |

TABLE 1B
Number of LTC-IC

| CB (10 donors) | Frequency of LTC-IC |
|---|---|
| Lin⁻CD34⁻CD38⁻ C1qRp⁻ | 1/4,000 |
| Lin⁻CD34⁻CD38⁻ C1qRp⁺ | 1/3,500 |
| Lin⁻CD34⁺CD38⁻ C1qRp⁺ | 1/16 |
| Lin⁻CD34⁺CD38⁻ C1qRp⁻ | 1/24 |

These results suggested that further fractionation of Lin⁻CD34⁻CD38⁻ based on C1qRₚ expression did not increase their clonogenic capacity. Moreover, the use of C1qRₚ as a positive selection marker did not enrich for more commited progenitor cells. To the contrary, both Lin⁻CD34⁺CD38⁻C1qRₚ⁺ and Lin⁻CD34⁺CD38⁻C1qRₚ cell populations were highly clonogenic (in both CFC and LTC-IC assays). Thus, the fractionation of Lin⁻CD34⁺CD38⁻ cells based on the expression of C1qRₚ did not appear to select one way or the other for clonogenic cells.

II. Human Lin⁻CD34⁻CD38⁻C1qRₚ⁺ Cells Contain SCID-repopulating Cells:

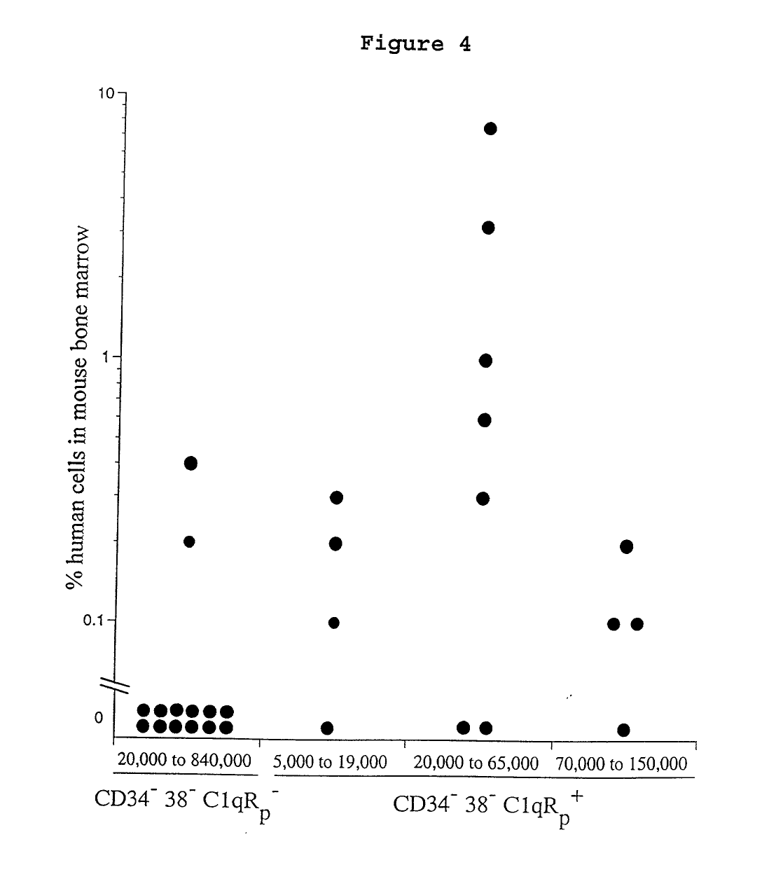

To determine whether the further purification of Lin⁻CD34⁻CD38⁻ based on C1qRₚ expression could enrich SCID-repopulating cell (SRC) activity and to determine the frequency of repopulating cells, purified cells were transplanted at varying cell doses into NOD/SCID mice using standard protocols. Mouse bone marrow was analyzed for the presence of human cells after 6-8 weeks. The levels of human cell engraftment in 29 NOD/SCID mice transplanted with umbilical cord blood Lin⁻CD34⁻CD38⁻C1qRₚ⁺ or Lin⁻CD34⁻CD38⁻C1qRₚ⁻ cell populations were quantified by flow cytometry and DNA analysis (FIG. 4). The majority of mice (12 out of 14) transplanted with Lin⁻CD34⁻CD38⁻C1qRₚ⁻ umbilical cord cells (dose ranging from 20,000 to 840,000 cells) were not engrafted (limit of detection: <0.05%). To the contrary, 11 out of 15 mice engrafted when transplanted with Lin⁻CD34⁻CD38⁻C1qRₚ⁺ cells indicating that the C1qRₚ⁺ subfractions were highly enriched in SRC. Transplantation with as few as 5,000 Lin⁻CD34⁻CD38⁻C1qRₚ⁺ cells derived from umbilical cord blood resulted in engraftment, whereas as many as 840,000 Lin⁻CD34⁻CD38⁻C1qRₚ⁻ cells were incapable of repopulation (FIG. 4). Previously, transplantation of at least 125,000 Lin⁻CD34⁻ cells was necessary to obtain engraftment. However, purification based on the expression of C1qRₚ⁺ provides more than a 25-fold enrichment in CD34^{neg}-SRC.

As shown in FIG. 3A, more than 99% of Lin⁻CD34⁺CD38⁻ cells expressed C1qRₚ. The presence of CD34P^{pos}-SRC activity in the Lin⁻CD34⁺CD38⁻C1qRₚ⁺ cell population was confirmed with a frequency similar to what was reported previously, i.e., 1 in 600 Lin⁻CD34⁺CD38⁻ cells (data not shown). Thus, the positive selection of human stem cells based on the expression of C1qRₚ allowed for the isolation of both CD34P^{pos}-SRC and CD4^{neg}-SRC.

### EXAMPLE 3

### Multi-parameters Analysis of Lin⁻CD34⁻CD38⁻C1qRₚ⁺ and Lin⁻CD34⁺CD38⁻C1qRₚ⁺ Cells

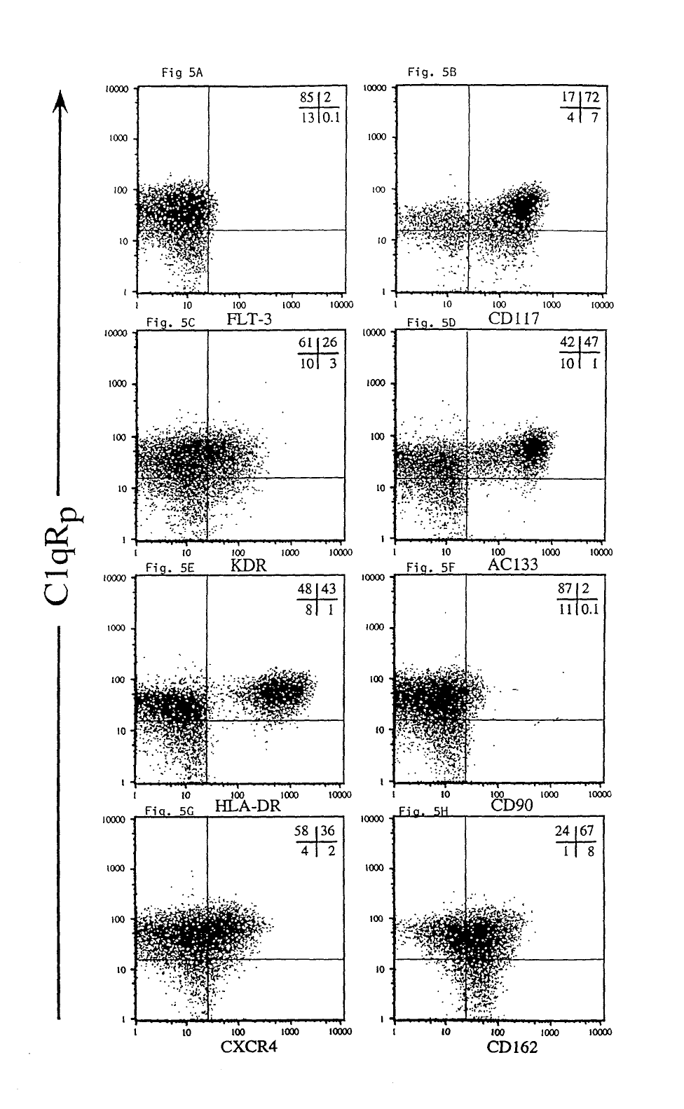

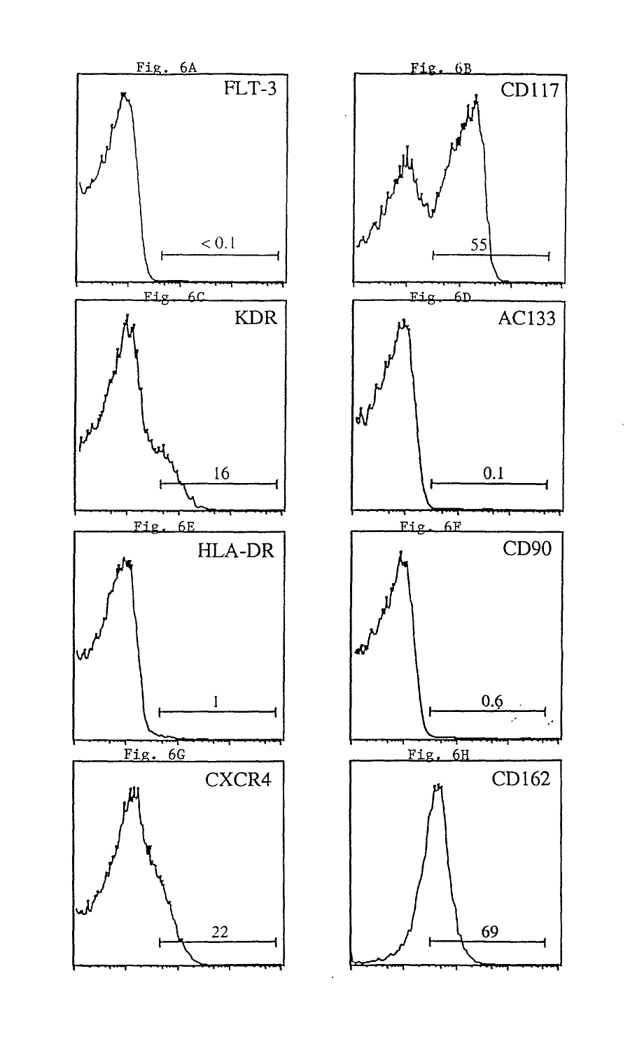

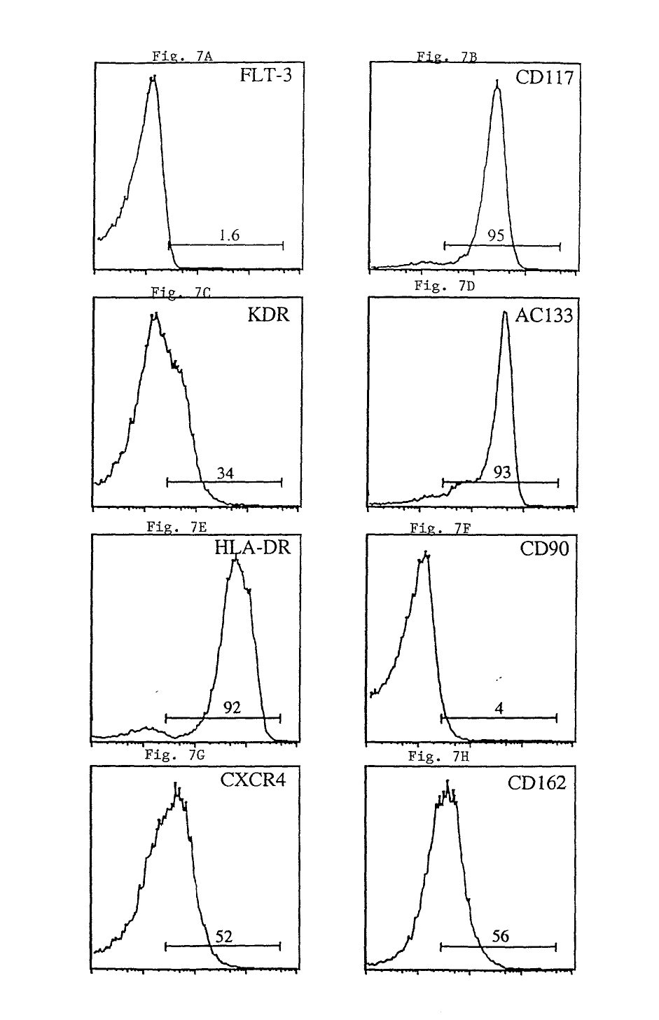

In order to determine whether other molecules associated with hematopoietic stem cells (KDR, CD117, HLA-DR, AC133, CD90) or stem cell homing (CXCR4 and CD162) were present on Lin⁻CD34⁻CD38⁻C1qRₚ⁺ and Lin⁻CD34⁺CD38⁻C1qRₚ⁺ cell populations, a four-color FACS analysis was performed. FIGS. 5A-5H show the results of one representative experiment from a pool of four umbilical cord blood samples. In FIGS. 6A-6H and FIGS. 7A-7H, the analysis was performed on gated Lin⁻CD34⁻CD38⁻C1qRₚ⁺ and Lin⁻CD34⁺CD38⁻C1qRₚ⁺ cells, respectively. The most important results from these analyses are the differences in expression levels of HLA-DR, and AC133 between Lin⁻CD34⁻CD38⁻C1qRₚ⁺ and Lin⁻CD34⁺CD38⁻C1qRₚ⁺ cells. Both cell populations also expressed KDR, CD117, CXCR4 and CD162 with only slight differences in expression levels.

### EXAMPLE 4

### RT-PCR Analysis of Cell Surface Antigen

Lin⁻CD34⁻CD38⁻C1qRₚ⁺ and Lin⁻CD34⁻CD38⁻C1qRₚ⁻ cell populations were purified, and mRNA was extracted to determine by RT-PCR the expression level of receptors implicated either in stem cell survival, homing and/or self-renewal. Table 2 below shows the representative results from five umbilical cord blood samples and one bone marrow sample. The expression of KDR in both cell subfractions confirmed the presence of the cell surface proteins observed by FACS.

TABLE 2
Expression of receptors implicated either in
stem cell survival, homing and/or self-renewal

| mRNA expression | Jagged-1 | LIF-R | KDR | FLT-3R | c-MPL |
|---|---|---|---|---|---|
| Lin⁻CD34⁻CD38⁻C1qRₚ⁻ | − | − | + | − | − |
| Lin⁻CD34⁻CD38⁻C1qRₚ⁺ | − | − | + | − | − |

### EXAMPLE 5

### C1qRₚ⁺ Cells Differentiate into Human Hepatocytes

Recent reports have indicated that the bone marrow of adult rodents contains cells that have the capacity to give rise to hepatocytes (21-23). Lagasse et al. established that some hematopoietic stem cells present in adult bone marrow co-purified with stem cells that gave rise to hepatocytes (2), which supports the hypothesis that somatic stem cells can change cell fate. However, these reports were based on adult rodent stem cell populations which may differ from human stem cells in their capacity to give rise to multiple tissues.

Studies by Theise et al. (7) and Alison et al. (8) based on patients who received bone marrow transplants, indicated that some human adult stem cells present in bone marrow also have the ability to give rise to hepatocytes. However, these two studies did not distinguish whether hematopoietic stem cells, mesenchymal stem cells or hepatocytes stem cells residing in the bone marrow were responsible for the liver engraftment.

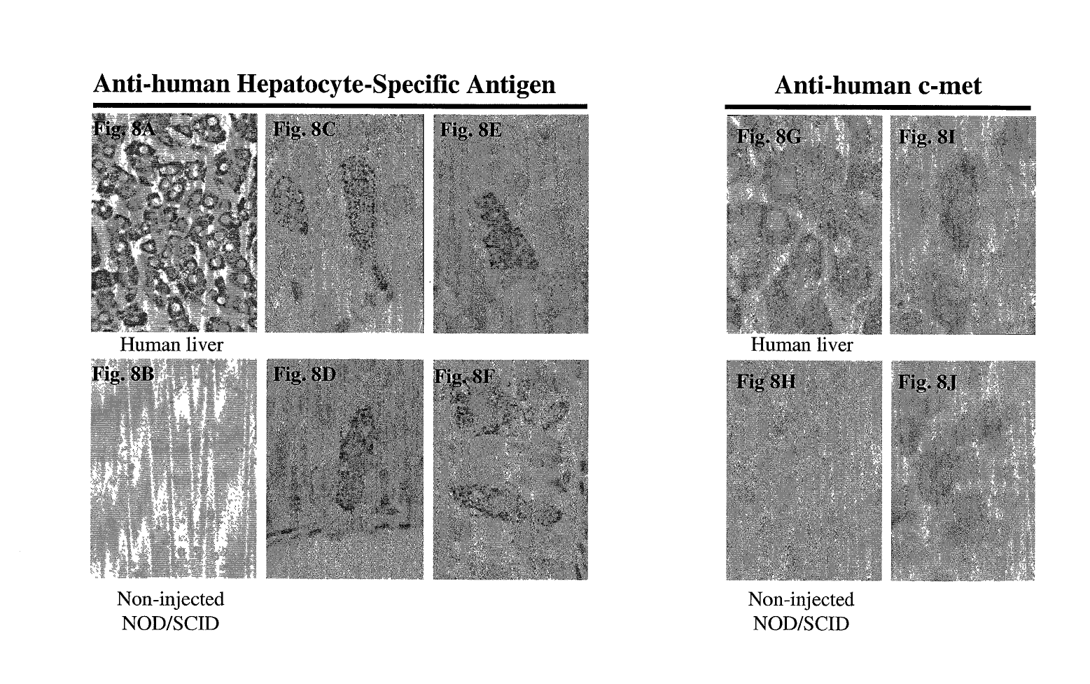

Thus, in order to determine whether C1qRₚ⁺ hematopoietic stem cells differentiate in vivo into hepatocytes, livers from sublethally irradiated NOD/SCID mice injected with either Lin⁻CD34⁻CD38⁻C1qRₚ⁺ or Lin⁻CD34⁺CD38⁻C1qRₚ⁺ cells were assessed by flow cytometry, immuno-histochemistry, and RNA expression analysis. Paraffin-embedded liver sections were immunostained for human hepatocyte-specific antigen (HSA), and c-met (hepatocyte growth factor receptor). Liver sections from non-injected NOD/SCID mice were used as negative controls. Both Lin⁻CD34⁻CD38⁻C1qRₚ⁺ and Lin⁻CD34⁺CD38⁻C1qRₚ⁺ cells gave rise to individual and/or clusters of human hepatocytes in mouse livers based on their morphology and positive staining for HSA (FIGS. 8c-8f) or c-met (FIGS. 8i and 8j).

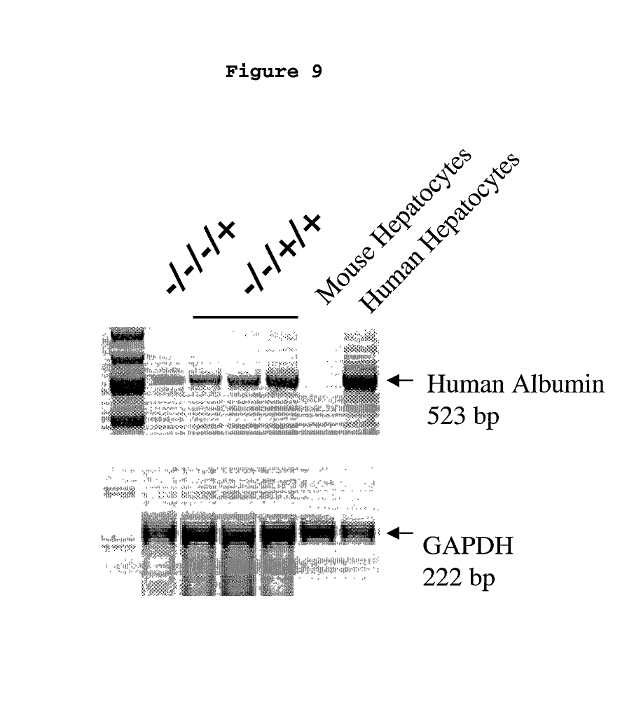

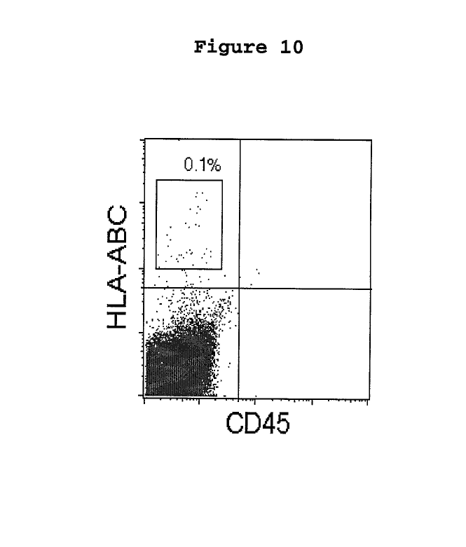

The presence of human hepatocytes and their ability to express a human hepatocyte-specific gene in the context of mouse liver tissue were confirmed by human albumin-specific RT-PCR (FIG. 9). The presence of human non-hematopoietic stem cells in the mouse liver was also confirmed by the detection of a rare population (≦0.1%) of human cells expressing human MHC class I molecules (HLA-ABC) which lacked the expression of CD45 (FIG. 10).

These data provide the first direct demonstration that a highly purified and phenotypically defined human stem cell population can repopulate the bone marrow and differentiate in vivo into functional hepatocytes. It is also one of the first demonstrations that, in the absence of overt tissue damage (i.e, sublethal irradiation), cell fate transitions can occur. The therapeutic potential of bone marrow is likely to be in the healing of liver after massive damage. Thus, the hematopoietic stem cells of the invention are suitable for the treatment of a wide variety of human disorders. In addition, the treatment of liver diseases with hematopoietic stem cells provides considerable advantages over the use of hepatocytes themselves.

The full developmental capacity of the new primitive C1qRₚ⁺ cell populations is still under investigation. It is highly likely that the C1qRₚ⁺ stem cells of the invention will retain the plasticity to differentiate into brain, keratinocytes, smooth muscle, cardiomyocyte, gut, lung and kidney epithelium.

In conclusion, the identification of C1qRₚ as a new marker for the most primitive human hematopoietic stem cells provides a new means to isolate and define this cell population. Moreover, the identification of C1qRₚ will facilitate the enrichment of CD34⁻ stem cells, which is required for their further characterization and ultimately for their use in medical procedures. Despite the lack of understanding of the mechanisms underlying stem cell plasticity, the results presented above suggest that human C1qRₚ⁺ hematopoietic stem cells provide a valuable source of stem cells for therapeutic bone marrow and/or liver repopulation.

### REFERENCES

1. Ashara T., Murohara T., Sullivan A., Silver M., et al. Isolation of putative progenitor endothelial cells for angiogenesis. Science, 1997, 254, 964-67.

2. Ferrari G., Cusella-De Angelis G., Coletta M., Paolucci et al. Muscle regeneration by bone-marrow derived myogenic progenitors. Science, 1998, 279, 1528-30.

3. Gussoni E., Soneoka Y., Strickland C. D., Buzney E. A., Khan M. K., Flint A. F., Kunkel L. M., Mulligan R. C. Dystrophin expression in the mdx mouse restored by stem cell transplantation. Nature. 1999, 401, 390-4.

4. Petersen B E., Bowen W C., Patrene K D. Mars W M., et al. Bone marrow as a potential source of hepatic oval cells. Science, 1999, 284, 1168-70.

5. Eglitis M A., and Mezey E. Hematopoietic cells differentiate into both microglia and macroglia in the brains of adult mice. Proc. Natl. Acad. Sci. USA, 1997, 94, 4080-85.

6. Reyes M. and Verfaillie C. Turning marrow into brain: Generation of glial and neuronal cells from adult bone marrow mesenchymal stem cells. Blood, 1999, 94, Abst. 1676, 377a.

7. Theise ND, Krause DS, Nimmakayalu M, Gardner R, Illei PB, Morgan G, Teperman L, and Henegariu O. Liver from bone marrow in humans. Hepatology, 2000, 32: 11-6.

8. Alison M R, Poulson R, Jeffery R, Dhillon A P, Quaglia A, Jacob J, Novelli M, Prentice G, Williamson J and Wright N A. Hepatocytes from non-hepatic adult stem cells. Nature, 2000, 406, 257.

9. Osawa M, Hanada K, Hamada H, Nakauchi H. Long-term lymphohematopoietic reconstitution by a single CD34− low/negative hematopoietic stem cell. Science. 1996; 273: 242-245.

10. Goodell M A, Rosenzweig M, Kim H, et al. Dye efflux studies suggest that hematopoietic stem cells expressing low or undetectable levels of CD34 antigen exist in multiple species. Nature Med. 1997; 3: 1337-1345.

11. Jones R J, Collector M I, Barber J P, et al. Characterization of mouse lymphohematopoietic stem cells lacking spleen colony-forming activity. Blood. 1996; 88: 487-491.

12. Donnelly DS, Zelterman D, Sharkis S, Krause D S. Functional activity of murine CD34+ and CD34− hematopoietic stem cell populations. Exp Hematol. 1999; 27:788-796.

13. Zanjani ED, Almeida-Porada G, Livingston A G, Flake A W, Ogawa M. Human bone marrow CD34− cells engraft in vivo and undergo multilineage expression that includes giving rise to CD34+ cells. Exp Hematol. 1998, 26: 353-360.

14. Bhatia M, Bonnet D, Murdoch B, Gan O, Dick JE. Identification of a newly discovered class of human hematopoietic cells with SCID-repopulating activity. Nature Med. 1998; 4: 1038-1044.

15. Petrenko O, Beavis A, Klaine M, Kittappa R, Godin I and Lemischka I R. The molecular characterization of the fetal stem cell marker AA4. Immunity, 1999; 10: 691-700.

16. Neponuceno R R, Henschen-Edman A H, Burgess W H and Tenner A J. CDNA cloning and primary structure analysis of C1qRₚ, the human C1q/MBL/SPA receptor that mediates enhanced phagocytosis in vitro. Immunity, 1997; 6: 119-29.

17. Morel F, Galy A, Chen B, Szilvassy S J: Equal distribution of competitive long-term repopulating stem cells in the CD34+ and CD34− fractions of Thy1lowLin/lowSca-1+ bone marrow cells. Exp Hematol. 1998; 26:440-445.

18. Goodell M A. CD34 (+) or CD34 (−): does-it matter? Blood. 1999; 94: 2548-2554.

19. Sato T, Laver J. H, Ogawa M. Reversible expression of CD34 by murine hematopoietic stem cells. Blood 1999; 94: 2548-2554.

20. MacKeam J P, McCubrey J, Fagg B. Enrichment of hematopoietic precursor cells and cloning of multipotential B lymphocyte precursors. Proc. Natl. Acad. Sci. USA. 1985; 82: 7414-18.

21. Petersen, B. E., Goff, J. P., Greenberger, J. S. & Michalopoulos, G. K. Hepatic oval cells express the hematopoietic stem cell marker Thy-1 in the rat. Hepatology 27, 433-45 (1998).

22. Lagasse, E. et al. Purified hematopoietic stem cells can differentiate into hepatocytes in vivo. Nat Med 6, 1229-34 (2000).

23. Theise, N. D. et al. Derivation of hepatocytes from bone marrow cells in mice after radiation-induced myeloablation. Hepatology 31, 235-40 (2000).

24. Krause, D. S., N. D. Theise, M. I. Collector, 0. Henegariu, S. Hwang, R. Gardner, S. Neutzel, and S. J. Sharkis. 2001. Multi-organ, multi-lineage engraftment by a single bone marrow-derived stem cell. Cell. 105:369-377.

25. Bjornson C R, Rietze R L, Reynolds B A, et al. Turning brain into blood: a hematopoietic fate adopted by adult neural stem cells in vivo. Science, 283: 534-538, (1999).

26. Eglitis M A and Mezey E. Hematopoietic cells differentiate into both microglia and thinking or therapeutic perspective? J Clin Invest, 105: 1669-1674, (2000).

27. Orlic D., Kajstura J., Chimenti S., Limana F., et al. Mobilized bone marrow repair the infarcted heart, improving function and survival. Proc. Acad. Sci. USA., 98: 10344-349.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of identifying the presence of human hematopoietic stem cells in a mixed cell population comprising exposing said cell population to an antibody or fragment thereof immunologically specific for C1qRₚ, the occurrence of anti-C1qRₚ binding being indicative of the presence of said human hematopoietic stem cells in said cell population.

2. The method of claim 1, wherein said anti-C1qRₚ bound cells are separated from non-anti-C1qRₚ bound cells.

3. Stem cells isolated using the method of claim 1.

4. The method of claim 1, wherein the human hematopoietic stem cells are CD34⁻.

5. The method of claim 1, wherein the human hematopoietic stem cells are CD34⁺.

6. The method of claim 1, wherein said antibody comprises a detectable label.

7. The method of claim 1, wherein the mixed cell population is obtained from a source selected from the group consisting of bone marrow cells and umbilical cord blood cells.

8. The method of claim 1 wherein step (b) comprises a selection step selected from the group consisting of fluorescence-activated cell sorting and magnetic bead separation.

9. The method of claim 1, wherein the antibody or fragment is in solution.

10. The method of claim 1 wherein said antibody or antibody fragment is immobilized on a solid support.

11. The method of claim 10, wherein the solid support is a magnetic bead.

12. The method of claim 1, wherein cells so isolated are further contacted with a second antibody immunologically specific for CD34.

13. The method of claim 1, wherein cells so isolated are further contacted with a second antibody immunologically specific for CD38.

14. The method of claim 1, wherein said mixed cell population is contacted with antibodies immunologically specific for lineage-related markers selected from the group consisting of CD2, CD3, CD14, CD16, CD19, CD24, CD56, CD66b, CD41 and glycophorin A prior to the performance of step a).

15. The method of claim 6, wherein said detectable label is selected from the group consisting of fluorescein, rhodamine, phycoerythrin, biotin, and strepavidin.

16. A method for obtaining human pluripotent stem cells, comprising:
a) obtaining a mixed cell population suspected of comprising pluripotent stem cells;
b) exposing said mixed cell population to an antibody or fragment thereof having binding affinity for C1qRₚ;
c) separating anti-C1qRₚ antibody bound cells from non anti-C1qRₚ antibody bound cells present in said mixed cell population thereby isolating C1qRₚ⁺ stem cells; and
d) resuspending said isolated C1qRₚ⁺ cells in a biologically compatible buffer.

17. The method of claim 16, whrein said mixed cell population is obtained from bone marrow cells or umbilical cord cells.

18. C1qRₚ⁺ cells isolated by the method of claim 16.

19. A method for repopulating a stem cell population in a host animal, said method comprising:
a) exposing said animal to agents which essentially eradicate said animal's existing stem cell population;
b) obtaining a population of C1qRₚ⁺ stem cells which have major histocompatiblity complex compatibility with said host animal and
c) transplanting said stem cells into said animal under conditions suitable for engraftment, thereby repopulating said stem cell population is said host animal.

20. The method of claim 19, wherein said host animal is a human.

21. The method of claim 19, wherein engraftment of said stem cells repopulates cells in both the hematopoietic and hepatocyte lineages.

22. The method of claim 19, wherein said C1qRₚ⁺ cells are CD34⁻.

23. The method of claim 16, wherein said cells form hematopoietic cell colony forming units when cultured on methylcellulose.
